(19) United States
(12) Patent Application Publication
(10) Pub. No.: US 2011/0172612 A1
(43) Pub. Date: Jul. 14, 2011

(54) Negative Pressure Wound Therapy Device

(76) Inventors: Bryan Greener, York (GB); John Hicks, York (GB); Edward Yerbury Hartwell, York (GB)

(21) Appl. No.: 13/000,580

(22) PCT Filed: Jun. 15, 2009
(86) PCT No.: PCT/GB2009/001486

§ 371 Date: Mar. 30, 2011

(30) Foreign Application Priority Data
Jun. 24, 2008 (GB) 0811572.7

(51) Int. Cl. A61F 13/00; A61M 1/00

(52) U.S. Cl. 604/313

(57) ABSTRACT

Apparatus for the application of topical negative pressure, or vacuum, therapy to a wound site.

This invention concerns the provision of a small portable negative pressure wound therapy device.

### BACKGROUND

Negative Pressure Wound Therapy devices currently on the market are large and not easily portable. These devices are often hospital based and cannot be moved without assistance from hospital staff. Even the smaller devices described as portable are still relatively large and cumbersome being highly obtrusive and noticeable. Making devices that are very smaller and unobtrusive has not been possible before without a loss of function and/or quality.

Many of the current Negative Pressure Wound Therapy devices are also inefficient due to having leakages around the seal of the wound letting in air and potentially harmful bacteria, fungi or other organisms. This often means that a larger, more powerful pump is needed to be able to cope with the extra pumping or suction required to maintain the vacuum or a negative pressure in the wound cavity as there is a constant leak. Stronger adhesives per se used on the dressings may help address this problem but are painful to the patient to remove and are usually very rigid and prone to cracking and thus ultimately leakage.

This present invention addresses these problems of the prior art. Surprisingly we have found the provision of a substantially leak-free interface dressing for use with current and future negative pressure therapy devices.

This invention concerns the provision of a substantially leak-free patient interface dressing (comprising for example a wound filler and a drape) for use with current and future negative pressure (or vacuum) wound therapy devices. Current patient interface methods and dressings result in high vacuum leak rates that necessitate compensation by the use of high fluid flow rate pumps.

Such pumps are relatively large and heavy. A further disadvantage is their need to have a powerful motor in order to provide the torque and power to drive the pump for long periods of time, and this, in turn, necessitates the provision of a power supply of significant power capacity in order to provide useful therapy life. All of these factors lead to current negative pressure wound therapy units being cumbersome, heavy, indiscreet and inconvenient for the patient. Surprisingly we have found that a key barrier to miniaturisation of current systems lies in the interface leak rate. This can be as high as several hundred millilitres of gas per minute, well in excess of the maximum fluid exudation rate under negative pressure (a few millilitres per minute at most). Leak rates are so high because of the mechanical mismatching of interface dressing to both the mechanical properties of skin and the geometry of the attachment site. Typical dressing systems include a wound cavity packing element (e.g. foam or gauze), a covering drape (typically a standard adhesive medical drape) and a means of introducing a vacuum tube into the cavity so enclosed. Such systems involve many components and the custom fitting of these components at the time of application; this is complex and leads to unavoidable inconsistencies and deviations from optimal patient care. The aim of this invention is the provision of a low leak rate negative pressure wound therapy dressing that is easy to apply and can be applied to a range of anatomical sites with high reproducibility. This invention will facilitate the development of miniaturised negative pressure wound therapy systems.

### SUMMARY OF INVENTION

This invention relates to the consistent provision of low leak rate patient interface dressings for negative pressure wound therapy. Low leak rates are here considered to be in the range of 0-50 ml/min. Consistent is taken to mean a success rate for achieving low leak rate in 90% or greater of applications to the patient.

The design of the patient interface dressing that is the subject of this invention relies, for its low leak rate, on a novel drape material. Here, drape is the term used to describe the continuous layer that encloses the vacuum cavity (which may optionally contain a filling element, separate from, attached to or integral to the drape). It would also be obvious to one skilled in the art that numerous configurations of this drape can be envisaged which would allow it to have other properties as well as acting as this enclosure layer. These could include exudates, absorbency, inclusion of an anti-microbial (e.g. silver salts) or other active agent (which may or may not be released from the dressing) or for example odour absorption.

According to a first aspect of the invention there is provided apparatus for the application of topical negative pressure or vacuum wound therapy to a wound site, the apparatus comprising:

a wound covering element (drape) that provides a substantially airtight seal over the wound site in which at least one portion of the wound covering element is flexible;

a vacuum connection tube (inlet tube) connecting a wound cavity to a vacuum source, and the vacuum source connected to a distal end of the vacuum connection tube, wherein the flexible portion of the wound covering element has substantially a similar flexibility or stretch as that of skin.

The drape of this invention may consist of a single element or several continuous or discontinuous elements. The drape when assembled has a force constant of less than 500 Nm⁻¹(Newton per metre), or less than 250 Nm⁻¹, or less than 150 Nm⁻¹, or less than 100 Nm⁻¹, or less than 50 Nm⁻¹, such that the drape is able to deform in accordance with deformation of the dermis in a mammal in both compression and extension. To achieve high extension at low applied force, the drape may have a thickness of less than 10 mm, preferably less than 5 mm, more preferably less than 3 mm.

For attachment to the patient, the drape may be constructed of an inherently adhesive material or have an adhesive layer bonded to its attachment surface. Peel force from intact skin is preferably less than 100 gfcm⁻¹ (grams force per centimetre), more preferably less than 50 gfcm⁻¹ such that the drape remains adhered to the skin whilst subjected to the stresses associated with tissue and the drapes ability to deform or force constant. In the case where an adhesive layer is applied, adhesive coat weight is preferably greater than 50 gm⁻² (grams per metre square), more preferably greater than 100 gm⁻² and even more preferably greater than 200 gm⁻², but should preferably not exceed 600 gm⁻².

The low force constant adhesive drapes of this invention can be constructed of any material but are preferably constructed of silicone or polyurethane based materials. When the subjects of this invention are constructed of silicone-based materials, this can include a proportion of silicone oil to enhance extensibility. The silicone oils have low viscosities ranging from 50 mPas to 750 mPas. Silicone oil can be added to the silicone elastomer formulation at any percentage, but preferably between 10% and 80% content, more preferably between 30% and 70% content and even more preferably between 35% and 55% content. They may be constructed of transparent, semi-transparent or opaque material but are preferably transparent or semitransparent. When the subjects of this invention are constructed of polyurethane based materials, they can include a proportion of organic plasticising agent for example trialkylacetyl citrates or dialkyl phthalates. They can also include a proportion of a plasticising glycol ether, e.g. polypropylene glycol dimethyl ether or similar. These plasticisers can be added to the polyurethane elastomer formulation preferably between 1 and 50% more preferably between 5 and 30% and most preferably between 10 and 20% content.

For application in negative pressure wound therapy, the drape is required to provide a low leak rate seal over a cavity containing sub-atmospheric pressure. The sub-atmospheric pressure is generated and replenished by a connected vacuum source, commonly a pump. The vacuum cavity is connected to the vacuum source by some means, commonly a flexible tube with single or multiple lumen. In some embodiments of the present invention a sufficiently small pump may be included in the dressing. In embodiments of the present invention the pump may be mechanically powered and/or battery and/or electrically powered. The tube can be connected to the vacuum cavity by any means and methods relying upon penetration through the drape layer and entrapment under the drape layer are both commonly applied in practice. In either case, the sealing means around the area of engagement is critical. The multi-component nature of current patient interfaces means that the connection of tubing with the vacuum cavity is application dependent and is a source of inconsistency. An embodiment of present invention would be manufactured as a single component, the drape and tube in these embodiments of the present invention would be integral, one part. For example the pump may be integral to the drape such that the inlet to the pump connects fluid directly into the cavity or via a short conduit to the cavity. In some embodiments of the present invention the apparatus of the present invention may be sprayed onto the patient or cast in situ on the patient. The present invention made be manufacture by molding or welding or gluing or any other known means.

A second aspect of this invention is the preferential incorporation of the vacuum connection means within the thickness of the drape as another method to reduce vacuum leakage. To facilitate this, the drape is profiled to a depth exceeding the vacuum connection means dimensions. The interface drape is constructed around the vacuum connection means and the connecting means preferable runs, sealed within the drape, from its edge to at least one, or a multiplicity of outlets in connection with the vacuum cavity. A device design of this kind overcomes a significant limitation of current drape and tubing designs, namely tubing induced drape displacement and subsequent vacuum leakage. Drape displacement occurs because of torque imparted from the tubing, either at the edge of the drape or at the centre of the drape, which is resultant from a single tubing attachment point. The drape-embedded vacuum connection tube that is one aspect of this invention is attached along the edge-to-centre dimension of the drape, effectively spreading the torque load generated over this dimension, therefore greatly reducing localised torque on the drape, thereby reducing the likelihood of drape leakage occurring by this mechanism. In other words it is harder to cause a leak from any pull on the tube in comparison to current products where the tube connection is under the drape with a connection point with a small area or through the drape also having a connection point with a small area between the tube coming into the wound cavity area and where it meets the drape. Prior art devices have the disadvantage that a tug on the tube may easily cause damage to the join area of the tube and drape resulting in leakage.

The present invention may have additional features like an irrigant conduit to the wound cavity, or means to monitor the pressure in the cavity e.g. remote wireless transducer or wired transducer. Embodiments of the present invention may also have a quick disconnect connector to conduits integral to the drape. The filler e.g. including but not limited to foam and/or gauze) used in the wound cavity may also be integral or not integral. In some embodiments the filler may be integral to the drape and inlet tube, and/or pump, drape and inlet tube. The filler may be bioresorbable. The apparatus of the present invention may be used on any type of wound, burn, or skin defect, including but not limited to grafts, flaps, dehisced incisions, chronic wounds, ulcers, surgical drains etc. The apparatus of the present invention may also contain a filter and/or a one way valve in the outlet tube.

The apparatus according to the second aspect of the invention can include some or all of the features outlined in the embodiments according to the first aspect of the invention.

Thus, according to a third aspect of the invention there is provided apparatus for the application of topical negative pressure or vacuum wound therapy to a wound site, the apparatus comprising:

a wound covering element (drape) that provides a substantially airtight seal over the wound site in which at least one portion of the wound covering element is flexible;

a vacuum connection tube (inlet tube) connecting a wound cavity to a vacuum source, and the vacuum source connected to a distal end of the vacuum connection tube, wherein the flexible portion of the wound covering element has substantially a similar flexibility or stretch as that of skin and wherein the vacuum connection means is incorporated within the thickness of the drape.

According to a fourth aspect of the invention there is provided an apparatus as substantially herein described with reference to the accompanying Examples and Figures.

The present invention will now be described by way of example with reference to the following drawings:

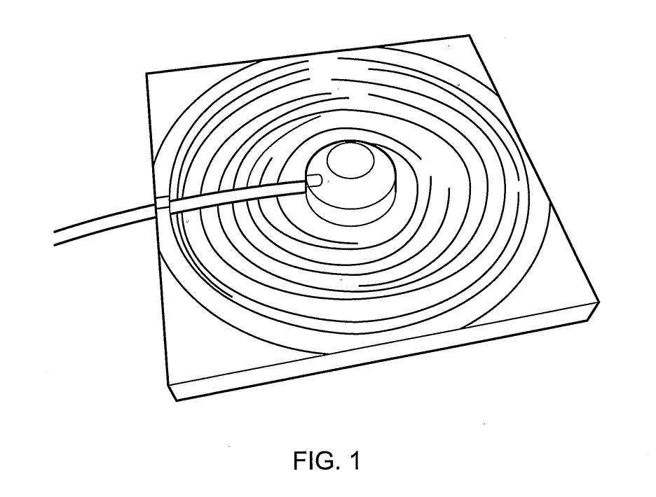

FIG. 1 shows an aluminium mould with silicone tubing in position for casting of heat curable silicone elastomer.

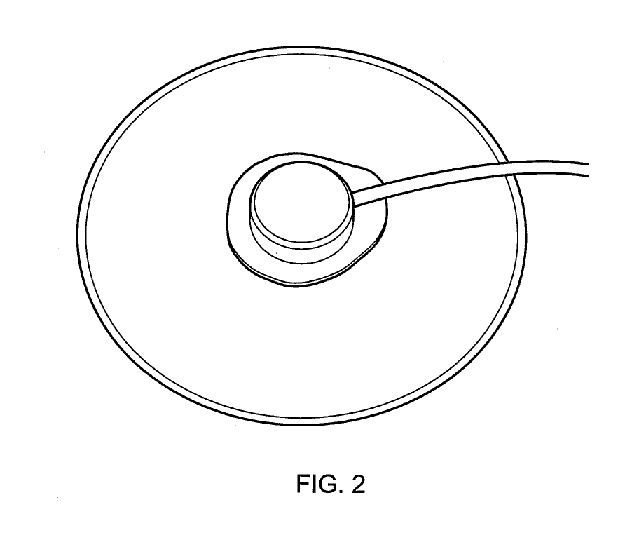

FIG. 2 shows a finished silicone drape with embedded vacuum connection tubing and silicone adhesive layer.

### EXAMPLES

### Example 1

A 4 mm outer diameter, 2 mm internal diameter silicone tube of circular cross section (Degania Silicone) was positioned in an aluminium mould (FIG. 1) and a two-part heat curable silicone elastomer (Wacker Silicones) was cast over the top of the tube. The resulting single-piece drape dressing was coated on its contact face with a two-part heat curable silicone elastomer adhesive (Dow Corning). The resulting drape with embedded vacuum connection tubing is shown in FIG. 2 and had a force constant of 500 Nm⁻¹.

### Example 2

A 4 mm outer diameter, 2 mm internal diameter silicone tube of circular cross section (Degania Silicone) was positioned in an aluminium mould (FIG. 1) and a two-part heat curable silicone elastomer (Wacker Silicones) with 40% w/w silicone oil (Aldrich Chemical Co.) added was cast over the top of the tube. The resulting single-piece drape dressing was coated on its contact face with a two-part heat curable silicone elastomer adhesive (Dow Corning). The resulting drape had a force constant of 100 Nm⁻¹, noticeably more extensible than the drape of Example 1 due to the addition of silicone oil.

### Example 3

A 4 mm outer diameter, 2 mm internal diameter silicone tube of circular cross section (Degania Silicone) was positioned in an aluminium mould (FIG. 1) and a two-part heat curable silicone elastomer (Wacker Silicones) with 50% w/w silicone oil (Aldrich Chemical Co.) added was cast over the top of the tube. The resulting single-piece drape dressing was coated on its contact face with a two-part heat curable silicone elastomer adhesive (Dow Corning). The resulting drape had a force constant of 50 Nm⁻¹, noticeably more extensible than the drape of Example 1 due to the addition of silicone oil. However, this drape had observably lower mechanical strength than those generated in Examples 1 and 2.

### Example 4

A 4 mm outer diameter, 2 mm internal diameter silicone tube of circular cross section (Degania Silicone) was positioned in a square polycarbonate mould of 30×30 cm dimensions. The tubing entered through the base of the midpoint of one side of the mould and ended in the centre. A two-part heat curable silicone elastomer (Wacker Silicones) with 40% w/w silicone oil (Aldrich Chemical Co.) added was cast over the top of the tube. The resulting single-piece drape dressing was coated on its contact face with a two-part heat curable silicone elastomer adhesive (Dow Corning). The resulting drape had a force constant of 100 Nm⁻¹ and was appropriate for low leak rate coverage of wounds of surface areas up to approximately 15×15 cm.

### Example 5

The drape of Example 2 was positioned above a 5 cm diameter gauze swatch (Kerlix AMD) attached to an intact human abdomen connected to a vacuum source regulated at 100 mmHg below ambient atmospheric pressure. The vacuum pump was monitored for 12 hours during which normal daily tasks were carried out (driving, sleeping, sitting, standing, walking etc). The pump, of <1 ml stroke capacity performed 2 strokes during this period, significantly less volume transmission than would occur from a wound.

### Example 6

The drape of Example 2 was positioned above a 5 cm diameter foam swatch (KCI) attached to an intact human abdomen connected to a vacuum source regulated at 100 mmHg below ambient atmospheric pressure. The vacuum pump was monitored for 12 hours during which normal daily tasks were carried out (driving, sleeping, sitting, standing, walking etc). The pump, of <1 ml stroke capacity performed 2 strokes during this period, significantly less volume transmission than would occur from a wound. Significant skin irritation occurred resulting from foam indentation on the skin.

1. Apparatus for the application of topical negative pressure or vacuum wound therapy to a wound site, the apparatus comprising:
a wound covering element that provides a substantially airtight seal over the wound site in which at least one portion of the wound covering element is flexible;
a vacuum connection tube connecting a wound cavity to a vacuum source, and the vacuum source connected to a distal end of the vacuum connection tube, wherein the flexible portion of the wound covering element has substantially a similar flexibility or stretch as that of skin.

2. An apparatus as claimed in claim 1, wherein the apparatus further comprises a wound contact element.

3. An apparatus as claimed in either claim 1, wherein the flexible portion of the wound covering element is the only portion of the wound covering element.

4. An apparatus as claimed in claim 1, wherein the flexible portion of the wound covering element has a flexibility or force constant of 50 Nm⁻¹ plus or minus that of skin.

5. An apparatus as claimed in claim 4, wherein the flexibility or force constant of the wound covering element (with and without an adhesive layer) is less than 500 Nm⁻¹.

6. An apparatus as claimed in claim 4, wherein the flexibility or force constant of the wound covering element (with and without an adhesive layer) is less than 250 Nm⁻¹.

7. An apparatus as claimed in claim 4, wherein in the flexibility or force constant of the wound covering element (with and without an adhesive layer) is less than 150 Nm⁻¹.

8. An apparatus as claimed in claim 4, wherein the flexibility or force constant of the wound covering element (with and without an adhesive layer) is less than 100 Nm⁻¹.

9. An apparatus as claimed in claim 1, wherein the flexibility or force constant of the wound covering element (with and without an adhesive layer) is less than 50 Nm ⁻¹.

10. An apparatus as claimed in claim 1, wherein the wound covering element has a thickness of less than 10 mm.

11. (canceled)

12. An apparatus as claimed in claim 10, wherein the wound covering element has a thickness of less than 5 mm.

13. An apparatus as claimed in claim 10, wherein the wound covering element has a thickness of less than 3 mm.

14. An apparatus as claimed in claim 1, wherein the wound covering element is constructed of an inherently adhesive material.

15. An apparatus as claimed in claim 1, further comprising an adhesive layer to detachably hold the wound covering element to the skin or wound site of the patient.

16. An apparatus as claimed in claim 1, wherein a peel force of the wound covering element (with or without an adhesive layer) detachably held to the skin or wound site of the patient is less than 100 gfcm.

17. An apparatus as claimed in claim 16, wherein the peel force of the wound covering element (with or without an adhesive layer) detachably held to the skin or wound site of the patient is less than 50 gfcm.

18. An apparatus as claimed in claim 1, wherein the wound covering element and the vacuum connection tube are integral.

19. An apparatus for the application of topical negative pressure or vacuum wound therapy to a wound site, the apparatus comprising:
a wound covering element that provides a substantially airtight seal over the wound site in which at least one portion of the wound covering element is flexible;
a vacuum connection tube (inlet tube) connecting a wound cavity to a vacuum source, and the vacuum source connected to a distal end of the vacuum connection tube, wherein the vacuum connection means is incorporated within the thickness of the wound covering element.

20. (canceled)
